(19) United States
(12) Patent Application Publication
(10) Pub. No.: US 2018/0311125 A1
(43) Pub. Date: Nov. 1, 2018

(54) COSMETIC COMPOSITIONS CAPABLE OF PRODUCING LOCALIZED SURFACE PLASMONIC RESONANCE IN RESPONSE TO INDOOR AND/OR OUTDOOR LIGHTING SOURCES

(71) Applicant: SABIC Global Technologies B.V., Bergen op Zoom (NL)

(72) Inventors: Heli Wang, Sugar Land, TX (US); Guangjun Yin, Shanghai (CN); Ihab N. Odeh, Sugar Land, TX (US)

(21) Appl. No.: 15/962,289

(22) Filed: Apr. 25, 2018

Related U.S. Application Data
(60) Provisional application No. 62/490,902, filed on Apr. 27, 2017.

(51) Int. Cl. A61K 8/19; A61N 5/06

(52) U.S. Cl. CPC A61K 8/19; A61N 5/062; A61K 2800/81; A61K 2800/413; A61N 2005/067; A61N 2005/0651; A61N 2005/0655; A61N 2005/0654; A61N 2005/0657

(57) ABSTRACT

A topical skin composition and methods for its use are described. The composition can include a plurality of first plasmonic noble metal nanostructures having an aspect ratio of greater than 1 and being configured to absorb electromagnetic radiation from an artificial light source and/or from a solar radiation source to produce a first localized surface plasmon resonance (LSPR), and a plurality of second plasmonic noble metal nanostructures having an aspect ratio of greater than 1 and being configured to absorb electromagnetic radiation from a solar radiation source to produce a second LSPR.

### CROSS REFERENCE TO RELATED APPLICATIONS

This application claims priority to U.S. Provisional Application Ser. No. 62/490,902 filed Apr. 27, 2017, which is incorporated by reference in its entirety without disclaimer.

### BACKGROUND OF THE INVENTION

### A. Field of the Invention

The invention generally concerns topical skin compositions that can be used for cosmetic or skin treatment applications. In particular, topical skin compositions of the present invention can be designed to produce a localized surface plasmon resonance (LSPR) in response to indoor lighting conditions and/or outdoor lighting conditions. The produced LSPR can be beneficial to skin (e.g., reducing anti-oxidants or free-radicals that can cause oxidative damage to skin cells).

### B. Description of Related Art

The use of plasmonic noble metal nanostructures in the cosmetics field is well-known. See WO 2012/027728 to Harris et al. Such structures have a plasmonic effect by producing a LSPR when subjected to electromagnetic radiation having a particular wavelength. For example, spherically-shaped plasmonic gold nanoparticles exhibit a LSPR in response to electromagnetic radiation having a wavelength range of about 510 nm to 530 nm, with a plasmonic peak of about 520 nm. See Huang and El-Sayed, Gold nanoparticles: Optical properties and implementations in cancer diagnosis and photothermal therapy, Journal of Advanced Research, Volume 1, Issue 1, pages 13-28 (2010). As explained by Huang and El-Sayed, when a plasmonic metal particle is exposed to electromagnetic radiation having a particular wavelength, the absorption of the electromagnetic radiation by the metal particle can induce oscillation of free conduction band electrons in the particle. This oscillation of the electrons around the particle surface results in a charge separation with respect to the ionic lattice, which forms a dipole oscillation along the direction of the electric field of the electromagnetic radiation. The amplitude of the oscillation reaches a maximum at a specific frequency (i.e., plasmonic peak or LSPR peak).

The plasmonic effect produced by plasmonic metal nanoparticles results in particles that can have unique optical properties. The cosmetics industry has leveraged these optical properties to produce a variety of cosmetic pigments based on such nanoparticles. By way of example, US Publication 2009/0022765 to Chung et al. discloses a cosmetic pigment that exhibits colors in the visible region. The pigment can include a mixture of gold nanoparticles that exhibit a red color, silver nanoparticles that exhibit a yellow color, gold-silver alloy nanoparticles that exhibit a flame color, and gold nanoparticles that exhibit a blue color. According to the authors in Chung et al., varying the amounts of each nanoparticle in a given composition can result in a desired color of the composition. Notably, however, the Chung et al. pigments fail to consider the stability of their optical properties when the light source varies. In particular, the pigments in Chung et al. can be prone to not exhibiting the desired colors when exposed to artificial light versus natural sunlight, thereby presenting a problem with obtaining a desired and consistent color irrespective of the light source. That is, the plasmonic effect of the Chung et al. nanoparticles may disappear when the lighting source changes.

In addition to optical properties, the plasmonic effect has also been used in skin treatment applications. By way of example, WO 2012/027728 to Harris et al. discloses cosmetic compositions that include a plurality of plasmonic nanoparticles in an amount effective to induce thermomodulation in a target tissue region with which the composition is topically applied. The thermomodulation is designed to damage, ablate, lyse, or denature skin cells or proteins contained in the target region as well as induce skin inflammation, activate heat shock proteins, or perturbate cell signaling or disrupt the cell micro environment in the target tissue region. Similar to Chung et al., Harris et al. also fails to explain whether the plasmonic nanoparticles can produce the thermomodulation effect irrespective of whether the composition having the nanoparticles exposed to natural or artificial light. Stated plainly, the compositions in Harris et al. fail to address a situation where the end user applying the composition may move from an environment that has natural light to an environment that largely includes artificial light (e.g., an office environment, a home, etc.) and unknowingly reduces the efficacy of the composition.

US Publication 2009/0326614 to El-Sayed et al. discloses the use of plasmonic nanoparticles to treat skin cancer via surface plasmon resonance properties of the particles. Similar to Harris et al., El-Sayed et al. also fails to address the situation where the light source may vary, thereby reducing the efficacy of the composition when the light source switches from an artificial light source to natural sunlight, and vice versa.

### SUMMARY OF THE INVENTION

A discovery has been made that provides a solution to at least some of the aforementioned problems associated with the use of plasmonic metal nanoparticles in topical skin care compositions such as cosmetic compositions. In particular, the solution is premised on the development and use of a combination of plasmonic metal nanostructures in topical skin care compositions. The nanostructures can be designed or structured to have specific plasmonic or LSPR peaks that can produce LSPR responses irrespective of whether the composition is subjected to different light sources. For example subjected to first light source (e.g., artificial light sources that are designed to focus on the visible light region such as fluorescent light, light emitting diode, a laser, etc.) and then subjected to a different light source (e.g., solar radiation such as natural sunlight, incandescent light, halogen light, or a solar simulator designed to simulate the solar spectrum). An advantageous technical effect of such a combination of nanostructures is that an end user, having the composition applied to their skin, can continue to receive the skin benefit properties of the composition when they are in an environment that has natural light and then move to an environment that largely includes artificial light (e.g., an office environment, a home, etc.) and vice versa. That is, the compositions of the present invention can produce therapeutically effective amounts of LSPR in a natural light setting as well as in an artificial light setting. The produced LSPR can be beneficial to skin (e.g., it can aid in reducing antioxidant and/or free-radical attack on skin cells). This beneficial effect can continue irrespective of whether the end user moves from a natural light setting (e.g., outdoors) to an artificial light setting (e.g., indoors), and vice versa, thereby allowing the end user to obtain a more consistent skin benefit from the compositions of the present invention.

Also discovered in the context of the present invention is a plasmonic noble metal nanostructure that can be tuned to produce an LSPR response in either artificial light conditions and/or solar radiation conditions. A plurality of such nanostructures can be incorporated into a topical skin composition, thereby making the composition capable of producing an LSPR response in both indoor settings (e.g., home, office, etc.) and outdoor settings (e.g., natural sunlight). The nanostructure can be designed to have a LSPR peak of about 580 nm to 650 nm, preferably about 600 nm to about 630 nm. As indicated above, however, additional nanostructures having different LSPR peaks can also be incorporated into the composition, thereby providing for a more robust LSPR response in both indoor and outdoor settings.

In one aspect of the present invention, there is disclosed a topical skin composition that can include (a) a plurality of first plasmonic noble metal nanostructures having an aspect ratio of greater than 1 and being configured to absorb electromagnetic radiation from an artificial light source or from a solar radiation source to produce a first localized surface plasmon resonance (LSPR), and optionally, (b) a plurality of second plasmonic noble metal nanostructures having an aspect ratio of greater than 1 and being configured to absorb electromagnetic radiation from a solar radiation source to produce a second LSPR. In some embodiments, the plurality of first plasmonic noble metals can be configured to absorb electromagnetic radiation having a wavelength of 580 nm to 650 nm, preferably 600 nm to 630 nm, to produce the first LSPR. The plurality of second plasmonic noble metals can be configured to absorb electromagnetic radiation having a wavelength of 651 nm to 750 nm, preferably 675 nm to 725 nm, or more preferably about 700 nm, to produce the second LSPR. Without wishing to be bound by theory, it is believed that this combination of nanoparticles can result in a composition that produces LSPRs in response to artificial light and solar radiation. In certain instances, the aspect ratio of the plurality of first plasmonic noble metal nanostructures can be 1.7 to 2.5, preferably about 1.9 to 2.3, and/or the aspect ratio of the plurality of second plasmonic noble metal nanostructures can be 2.5 to 3.5, preferably about 3.0. The compositions of the present invention can also include a plurality of third, fourth, fifth, sixth, seventh, eighth, ninth, or more plasmonic noble metal nanostructures. In one instance, the composition can include a plurality of third plasmonic noble metal nanostructures having an aspect ratio of greater than 1 and being configured to absorb electromagnetic radiation having a wavelength of 751 to 900 nm, preferably about 850 nm, to produce a third LSPR. The plurality of third plasmonic noble metal nanostructures can have an aspect ratio of 3.5 to 5.0, preferably about 4.5. Without wishing to be bound by theory, it is believed that by modifying the aspect ratios of the nanostructures, the plasmonic peaks of these nanostructures can be tuned to a particular electromagnetic radiation wavelength. In particular, it is believed that by increasing the aspect ratios, the plasmonic peaks can be shifted towards longer wavelengths. Conversely, by decreasing the aspect ratios, the plasmonic peaks can be shifted towards shorter wavelengths. This allows specific tuning or designing of the types of nanostructures that can be included in the compositions of the present invention to achieve a desired match or correlation to a particular light source or lighting sources. For example, a composition of the present invention can be tuned to produce an LSPR in response to (1) a light emitting diode (LED) source and solar radiation, (2) a LED source and a fluorescent light source, (3) a LED source and an incandescent light source, (4) a LED source and a halogen light source; (5) a LED source and a laser source; (6) a LED source, solar radiation, and a fluorescent light source, (7) a LED source, a fluorescent light source, and an incandescent light source, (8) a LED source, solar radiation, a fluorescent light source, and an incandescent light source, (9) a LED source, a solar radiation, a fluorescent light source, and a halogen light source, or (10) or any other combination of a LED source, a solar radiation source, a fluorescent light source, and/or a laser source.

In certain aspects of the present invention, the plurality of first, second, third, and/or more plasmonic noble metal nanostructures can be made from any noble metal. In preferred instances, the noble metal is gold, silver, platinum, or palladium nanostructures, or alloys thereof. In even more preferred instances, the plasmonic nanostructures are plasmonic gold nanostructures. The shapes of the nanostructures can vary with the preferred shape being a cylindrical/nanorod shape. In instances where a nanorod shape is used, two LSPRs per nanorod can be produced, one in the horizontal direction (transverse axis) and one in the vertical direction (longitudinal axis). The aforementioned LSPR/plasmonic peaks are in relation to the longitudinal axis LSPRs for nanorods.

The topical skin compositions can be structured in a variety of ways and include a variety of cosmetically acceptable ingredients, including those disclosed throughout the specification. In one non-limiting embodiment, the composition can be structured as an emulsion (e.g., water-in-oil emulsion, oil-in-water-emulsion, silicone-in-oil emulsion, oil-in-silicone emulsion, etc.). In some instances, the composition can be a lotion or a cream. In other instances, the composition can be an ointment. In further aspects, the composition can be a solution, a gel, a suspension, or an anhydrous powder or stick. Further, the compositions of the present invention can include an effective amount of the first, second, and/or third or more plurality of plasmonic metal nanostructures. An effective amount can be 0.0001 wt. % to 20 wt. %, preferably 0.001 wt. % to 5 wt. %, or more preferably 0.005 wt. % to 2 wt. %, of the first, second, and/or third or more plurality of plasmonic noble metal nanostructures, based on the total weight of the composition. Larger amounts such as 25 wt. %, 30 wt. %, 40 wt. %, 50 wt. %, or more are also contemplated in the context of the present invention.

Also disclosed in the context of the present invention is a method for topically applying any one of the compositions to skin. In certain instances, the composition can be subjected to electromagnetic radiation from an artificial light source and/or from a solar radiation source. The plurality of first plasmonic noble metal nanostructures can produce a first LSPR in response to the light source. In instances where the composition includes a plurality of second, third, or more plasmonic metal nanostructures, the composition can also produce second, third, or more LSPR(s) in response to targeted light sources that match the plasmonic peaks of the nanostructures. Matching the plasmonic peaks of the nanostructures to a targeted light source(s) means that the targeted light source(s) has a light spectrum at a particular wavelength or wavelength range that can trigger a LSPR response in any given nanostructure of the present invention. By way of example, any given LED light source may emit electromagnetic radiation over a broad range of the visible light spectrum. However, the intensity of that radiation may be stronger at a particular wavelength range (e.g., 600 nm to 630 nm) when compared with another range (e.g., 675 nm to 700 nm). By designing a nanostructure that can produce a LSPR response to this particular LED light source, that nanostructure can be tuned to have a plasmonic peak within the 600 nm to 630 nm range. Thus, the plasmonic peak of any given nanostructure of the present invention can be tuned to correlate to a light spectrum of a targeted light source. By way of example, it is possible to produce a nanostructure that produces a LSPR in response to a LED light source but not in response to a fluorescent source, and vice versa. As explained in more detail below, this tuning capability can be implemented by varying the aspect ratio of any given nanostructure of the present invention. By including various tuned nanostructures in a topical skin composition, LSPRs can continually be produced even in instances where the light sources change. As discussed in detail below, the produced LSPR(s) can be beneficial to skin in a variety of manners. One such benefit could be the reduction of free radicals and or oxidants, which can help reduce oxidation of skin cells or contents within the skin cells (e.g., proteins, nucleic acids, lipid bilayers, etc.).

In one aspect of the invention, 20 embodiments are described. Embodiment 1 is a topical skin composition comprising: (a)a plurality of first plasmonic noble metal nanostructures having an aspect ratio of greater than 1 and being configured to absorb electromagnetic radiation from an artificial light source and/or from a solar radiation source to produce a first localized surface plasmon resonance (LSPR); and (b) a plurality of second plasmonic noble metal nanostructures having an aspect ratio of greater than 1 and being configured to absorb electromagnetic radiation from a solar radiation source to produce a second LSPR. Embodiment 2 is the topical skin composition of embodiment 1, wherein: the plurality of first plasmonic noble metals are configured to absorb electromagnetic radiation having a wavelength of 580 nm to 650 nm to produce the first LSPR; and the plurality of second plasmonic noble metals are configured to absorb electromagnetic radiation having a wavelength of 651 nm to 750 nm to produce the second LSPR. Embodiment 3 is the topical skin composition of embodiment 2, wherein: the plurality of first plasmonic noble metals are configured to absorb electromagnetic radiation having a wavelength of 600 nm to 630 nm to produce the first LSPR; and the plurality of second plasmonic noble metals are configured to absorb electromagnetic radiation having a wavelength of 675 to 725, preferably about 700 nm, to produce the second LSPR. Embodiment 4 is the topical skin composition of any one of embodiments 2 to 3, wherein: the aspect ratio of the plurality of first plasmonic noble metal nanostructures is 1.7 to 2.5, preferably about 1.9 to 2.3; and the aspect ratio of the plurality of second plasmonic noble metal nanostructures is 2.5 to 3.5, preferably about 3.0. Embodiment 5 is the topical skin composition of any one of embodiments 1 to 4, further comprising a plurality of third plasmonic noble metal nanostructures having an aspect ratio of greater than 1 and being configured to absorb electromagnetic radiation having a wavelength of 751 to 900 nm, preferably about 850 nm, to produce a third LSPR. Embodiment 6 is the topical skin composition of embodiment 5, wherein the aspect ratio of the plurality of third plasmonic noble metal nanostructures is 3.5 to 5.0, preferably about 4.5. Embodiment 7 is the topical skin composition of any one of embodiments 1 to 6, further comprising: a plurality of fourth plasmonic noble metal nanostructures having an aspect ratio of greater than 1 and being configured to absorb electromagnetic radiation having a wavelength of 901 to 1150 nm, preferably about 1000 nm to 1100 nm, to produce a fourth LSPR, wherein the aspect ratio of the plurality of fourth plasmonic noble metal nanostructures is 5.0 to 7.5, preferably about 6.0 to 7.0; plurality of fifth plasmonic noble metal nanostructures having an aspect ratio of greater than 1 and being configured to absorb electromagnetic radiation having a wavelength of 1151 to 1400 nm, preferably about 1200 nm to 1300 nm, to produce a fifth LSPR, wherein the aspect ratio of the plurality of fifth plasmonic noble metal nanostructures is 7.5 to 10.2, preferably about 8.0 to 9.2; a plurality of sixth plasmonic noble metal nanostructures having an aspect ratio of greater than 1 and being configured to absorb electromagnetic radiation having a wavelength of 1401 to 1800 nm, preferably about 1500 nm to 1600 nm, to produce a sixth LSPR, wherein the aspect ratio of the plurality of sixth plasmonic noble metal nanostructures is 10.2 to 14.5, preferably about 11.2 to 12.5; or combinations thereof. Embodiment 8 is the topical skin composition of any one of embodiments 1 to 7, wherein the plurality of first plasmonic noble metal nanostructures are configured to absorb electromagnetic radiation from an artificial light source selected from a fluorescent light source, a light emitting diode light source, or a laser source, or a mixture thereof. Embodiment 9 is the topical skin composition of any one of embodiments 1 to 8, wherein the solar radiation source is natural sunlight, a solar simulator, an incandescent light source, or halogen light source. Embodiment 10 is the topical skin composition of any one of embodiments 1 to 8, wherein the plurality of first, second, third, fourth, fifth, and/or sixth plasmonic noble metal nanostructures are gold, silver, or platinum plasmonic nanostructures, or alloys thereof, preferably plasmonic gold nanostructures. Embodiment 11 is the topical skin composition of any one of embodiments 1 to 10, wherein the plurality of first, second, third, fourth, fifth, and sixth plasmonic noble metal nanostructures are nanorods. Embodiment 12 is the topical skin composition of embodiment 11, wherein the first, second, third, fourth, fifth and/or sixth LSPR(s) are produced along the longitudinal axis of the first, second, third, fourth, fifth and/or sixth plasmonic noble metal nanostructures, respectively. Embodiment 13 is the topical skin composition of any one of embodiments 1 to 12, wherein the composition is an emulsion, preferably an oil-in-water emulsion. Embodiment 14 is the topical skin composition of any one of embodiments 1 to 12, wherein the composition is a cream, a lotion, a solution, a gel, a suspension, or an anhydrous powder or stick. Embodiment 15 is the topical skin composition of any one of embodiments 1 to 14, wherein the composition comprises 0.0001 wt. % to 20 wt. %, preferably 0.001 wt. % to 5 wt. %, or more preferably 0.005 wt. % to 2 wt. %, of the first, second, third, fourth, fifth and/or sixth plurality of plasmonic noble metal nanostructures, based on the total weight of the composition.

Embodiment 16 is a method comprising topically applying the composition of any one of embodiments 1 to 15 to skin. Embodiment 17 is the method of embodiment 16, wherein the composition is subjected to electromagnetic radiation from: an artificial light source and/or from a solar radiation source and the plurality of first plasmonic noble metal nanostructures produces a first LSPR; and/or a solar radiation source and the plurality of second plasmonic noble metal nanostructures produces a second LSPR, the plurality of third plasmonic noble metal nanostructures produces a third LSPR, the plurality of fourth plasmonic noble metal nanostructures produces a fourth LSPR, the plurality of fifth plasmonic noble metal nanostructures produces a fifth LSPR, and/or the plurality of sixth plasmonic noble metal nanostructures produces a sixth LSPR, wherein the produced first and/or second and/or third LSPR(s) reduce(s) free radicals and/or oxidants. Embodiment 18 is the method of any one of embodiments 16 to 17, wherein: the aspect ratio of the plurality of first plasmonic noble metal nanostructures is 1.7 to 2.5, preferably about 1.9 to 2.3, and wherein the nanostructures absorb electromagnetic radiation having a wavelength of 580 nm to 650 nm, preferably 600 to 630 nm to produce the first LSPR; and/or the aspect ratio of the plurality of second plasmonic noble metal nanostructures is 2.5 to 3.5, preferably about 3.0, and wherein the nanostructures absorb electromagnetic radiation having a wavelength of 651 nm to 750 nm, preferably 675 nm to 725 nm, or more preferably about 700 nm, to produce the second LSPR; the aspect ratio of the plurality of third plasmonic noble metal nanostructures is 3.5 to 5.0, preferably about 4.5, and wherein the nanostructures absorb electromagnetic radiation having a wavelength of 751 nm to 900 nm, preferably about 850 nm, to produce the third LSPR; the aspect ratio of the plurality of fourth plasmonic noble metal nanostructures is 5.0 to 7.5, preferably about 6.0 to 7.0, and wherein the nanostructures absorb electromagnetic radiation having a wavelength of 901 nm to 1150 nm, preferably about 1000 nm to 1100 nm, to produce the fourth LSPR; the aspect ratio of the plurality of fifth plasmonic noble metal nanostructures is 7.5 to 10.2, preferably about 8.0 to 9.2, and wherein the nanostructures absorb electromagnetic radiation having a wavelength of 1151 nm to 1400 nm, preferably about 1200 nm to 1300 nm, to produce the fifth LSPR; and/or the aspect ratio of the plurality of sixth plasmonic noble metal nanostructures is 10.2 to 14.5, preferably about 11.2 to 12.5, and wherein the nanostructures absorb electromagnetic radiation having a wavelength of 1401 nm to 1800 nm, preferably about 1500 nm to 1600 nm, to produce the sixth LSPR. Embodiment 19 is the method of any one of embodiments 16 to 18, wherein the artificial lighting source is a fluorescent light source, a light emitting diode light source, or a laser source, or a mixture thereof. Embodiment 20 is the method of any one of embodiments 16 to 19, wherein the solar radiation source is natural sunlight, a solar simulator, an incandescent light source, or a halogen light source.

The following includes definitions of various terms and phrases used throughout this specification.

“Nanostructure” refers to an object or material in which at least one dimension of the object or material is equal to or less than 1000 nm (e.g., one dimension is 1 to 1000 nm in size). In a particular aspect, the nanostructure includes at least two dimensions that are equal to or less than 1000 nm (e.g., a first dimension is 1 to 1000 nm in size and a second dimension is 1 to 1000 nm in size). In another aspect, the nanostructure includes three dimensions that are equal to or less than 1000 nm (e.g., a first dimension is 1 to 1000 nm in size, a second dimension is 1 to 1000 nm in size, and a third dimension is 1 to 1000 nm in size).

In one embodiment, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

“Topical application” means to apply or spread a composition onto the surface of lips or keratinous tissue. “Topical skin composition” includes compositions suitable for topical application on skin and/or keratinous tissue. Such compositions are typically dermatologically acceptable in that they do not have undue toxicity, incompatibility, instability, allergic response, and the like, when applied to skin and/or keratinous tissue. Topical skin care compositions of the present invention can have a selected viscosity to avoid significant dripping or pooling after application to skin and/or keratinous tissue.

“Keratinous tissue” includes keratin-containing layers disposed as the outermost protective covering of mammals and includes, but is not limited to, lips, skin, hair, and nails.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or any variation of these terms includes any measurable decrease or complete inhibition to achieve a desired result (e.g., reduction of oxidation in skin cells, reduction in cytokines associated with skin inflammation such as TNF-α, interleukin-1, interleukin-2, interleukin-6, interleukin-8, etc.). The terms “promote” or “increase” or any variation of these terms includes any measurable increase in production of a substance (e.g., matrix proteins such as fibronectin, laminin, collagen, or elastin, molecules such as hyaluronic acid) triggered to achieve a desired result.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “effective,” as that term is used in the specification and/or claims, means adequate to accomplish a desired, expected, or intended result.

The terms “a” or “an” when used in conjunction with any of the terms “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. With respect to the transitional phase “consisting essentially of,” in one non-limiting aspect, a basic and novel characteristic of the compositions of the present invention is their ability to produce a LSPR in response to an artificial light source and/or a solar radiation source.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 provides a comparison of spherically shaped nanoparticles and nanorods (absorbance was normalized to that of 30 nm for nanoparticles at 400 nm). NPS is nanoparticles and NRS is nanorods.

FIG. 2 depicts a light spectra for commercial LED lights sources and a fluorescent light source.

FIG. 3 depicts graphs of relative light intensity versus wavelength for different commercial light sources.

FIG. 4 depicts the ASTM G173-03 reference solar spectrum.

While the invention is susceptible to various modifications and alternative forms, specific embodiments thereof are shown by way of example in the drawings. The drawings may not be to scale.

### DETAILED DESCRIPTION OF THE INVENTION

The topical skin formulations of the present invention can include a plurality of first, second, third, or more plasmonic noble metal nanostructures. Each plurality of nanostructures can have an aspect ratio of greater than one (1), which allows for the tuning of the nanoparticles to have desired plasmonic peaks (plasmonic peak and LSPR peak can be used interchangeably throughout this specification). By having blends of different plasmonic nanostructures (and therefore blends of different plasmonic peaks) in any given topical skin formulation, the resulting formulation can continue to produce LSPR(s) in response to different lighting conditions. Thus, the topical skin formulations of the present invention can be specifically tuned to match desired lighting conditions. This can allow for compositions of the present invention to produce LSPRs in response to specific lighting sources (e.g., artificial light sources and/or natural lighting sources such as sunlight). A technical advantage of this when compared with known cosmetic compositions is that the composition can continue to produce therapeutically effective amounts of LSPR irrespective of whether the lighting conditions change (e.g., change from LED lighting to incandescent light or change from artificial lighting to natural sunlight and vice versa.). In one preferred embodiment, it has been discovered that the creation of plasmonic nanostructures having a plasmonic peak of 600 nm to 630 nm can be used for both indoor lighting sources (LED, incandescent, fluorescent, laser, etc.) and outdoor lighting sources (e.g., natural sunlight or light from a solar simulator) to produce a desired LSPR. Even further, it has also been discovered that nanostructures having a plasmonic peak of 651 nm to 750 nm, preferably 675 nm to 725 nm, or more preferably about 700 nm, work well in response to outdoor lighting conditions (e.g., natural sunlight, incandescent light, halogen lights or solar simulator). Still further, it has been discovered that nanostructures having a plasmonic peak of 751 nm to 900 nm, preferably 850 nm, also works well in outdoor lighting conditions. By including various blends/combinations of such nanostructures in any given topical skin composition, the composition can continue to produce a robust LSPR response under changing lighting conditions (e.g., moving from outdoors to indoors or indoors to outdoors or a change from LED light to incandescent light, etc.).

These and other non-limiting aspects of the present invention are discussed in further detail in the following sections with reference to the Figures.

### A. Plasmonic Noble Metal Nanostructures

The oscillations of electrons in the conduction band of noble metal nanostructures can induce oscillating electric fields on the surface of the nanostructures. These oscillating electric fields produce a LSPR. The LSPR is triggered when a nanostructure absorbs a sufficient amount of light having a given wavelength, thereby resulting in the oscillation of the electrons. For any given nanostructure capable of producing a LSPR, there is a corresponding light absorbance spectrum that triggers the LSPR response. This absorbance spectrum is generally referred to as the plasmonic or LSPR peak.

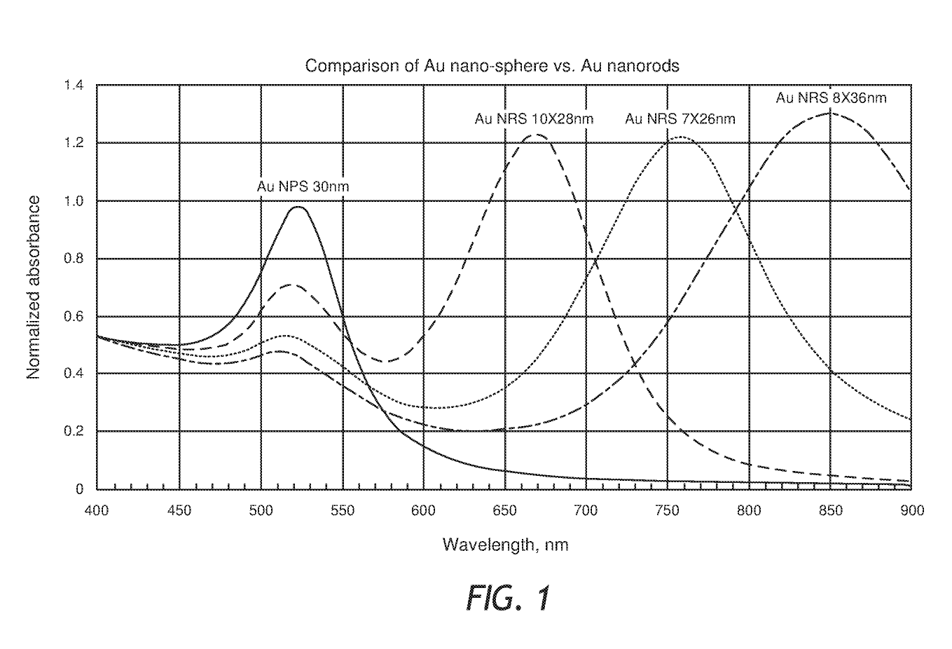

Plasmonic noble metal nanostructures can be designed or tuned to have a desired plasmonic peak. In particular, this tuning can occur by modifying the shape of the nanostructure to have a particular aspect ratio. “Aspect ratio” refers to the length divided by the width of a nanostructure. A length and a width of a given nanostructure can each be a straight-line distance measured between outermost surfaces of the nanostructure, where the length is the largest such distance and the width is perpendicular to the length. FIG. 1 illustrates the tuning concept guided by modifying the aspect ratio of a plasmonic gold nanostructure. In particular, the plasmonic peaks of gold spherical nanoparticles (NPS) are compared to gold rod-shaped nanostructures (NRS). The NPS, which has an aspect ratio of about 1, has a plasmonic peak of around 520 nm. By comparison, the peaks shift towards longer wavelengths (from higher to lower energy wavelengths or from blue light to red light and infrared light) when their respective aspect ratios are increased. For instance, gold NRS having an aspect ratio of 2.8 have a plasmonic peak of about 670 nm. Gold NRS having an aspect ratio of 3.71 have a plasmonic peak of about 760 nm. Gold NRS having an aspect ratio of 4.5 have a plasmonic peak of about 850 nm. Therefore, by changing the dimensions of the nanostructures, the nanostructures can be tuned to produce LSPRs at selected wavelengths having sufficient light intensities. Thus, combinations/blends/mixtures of plasmonic nanostructures can be created in the context of the present invention that can be used to produce therapeutically effective LSPRs in response to particular lighting conditions. By way of example only, a combination of plasmonic noble metal nanostructures can include: (1) a plurality of a first set of nanostructures that produce a first LSPR in response to any given artificial light source (e.g., light emitting diodes, fluorescent lights, lasers, etc.) or solar radiation source (natural sunlight, incandescent lights, halogen lights, or a solar simulator); and (2) a plurality of a second set of nanostructures that the produce a second LSPR in response to any given artificial light source or a solar radiation source. Combinations can include three, four, five, six, seven, or more sets of nanostructures each tuned to a given light wavelength and intensity, and hence, a given light source. This allows for the creation of combinations that can produce LSPRs in response to any combination of light sources. For example, a combination of plasmonic nanostructures can produce LSPRs in response to a light emitting diode (LED) source and solar radiation. Another combination of plasmonic nanostructures can produce LSPRs in response to a LED source and a fluorescent light source. A further combination of plasmonic nanostructures can produce LSPRs in response to a LED source and an incandescent light source. Another combination of plasmonic nanostructure can produce LSPRs in response to a LED source and a laser source or a halogen source. Yet another combination of plasmonic nanostructures can produce LSPRs in response to a LED source, solar radiation, and a fluorescent light source. Still further, another combination of plasmonic nanostructures can produce LSPRs in response to a LED source, a fluorescent light source, and an incandescent light source. Another combination of plasmonic nanostructures can produce LSPRs in response to a LED source, solar radiation, a fluorescent light source, and an incandescent light source. In the context of the present invention, any combination of nanoparticles can be created such that the resulting combination produces LSPRs in response to any desired lighting source. Further, and as discussed below and throughout this specification, these combinations can be incorporated into topical skin compositions, thereby resulting in compositions can produce effective LSPRs in response to changing lighting conditions (e.g., a user having the composition on skin moves from indoors to outdoors, or vice versa, or changes artificial lighting sources (e.g., LED to incandescent, or vice versa)).

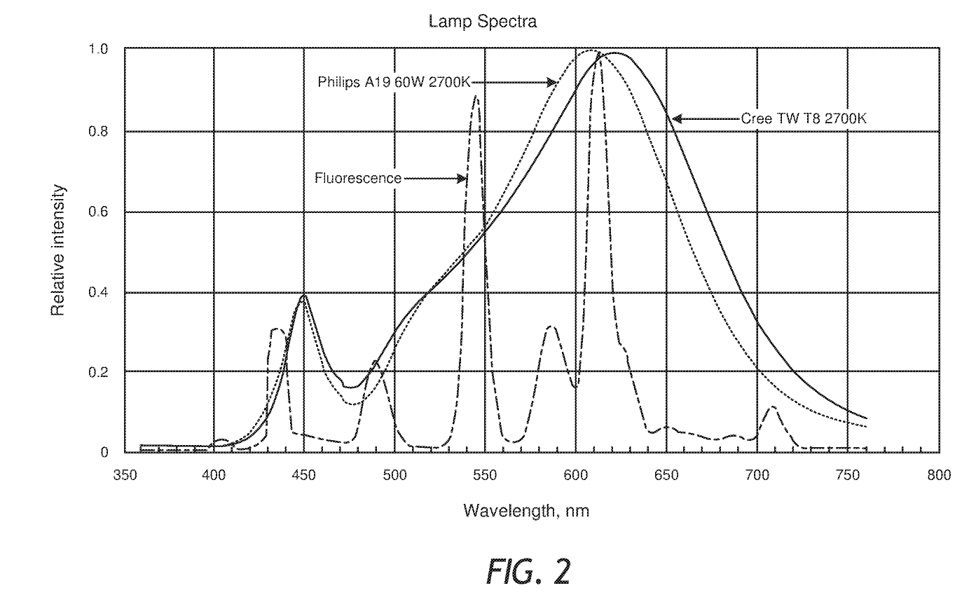

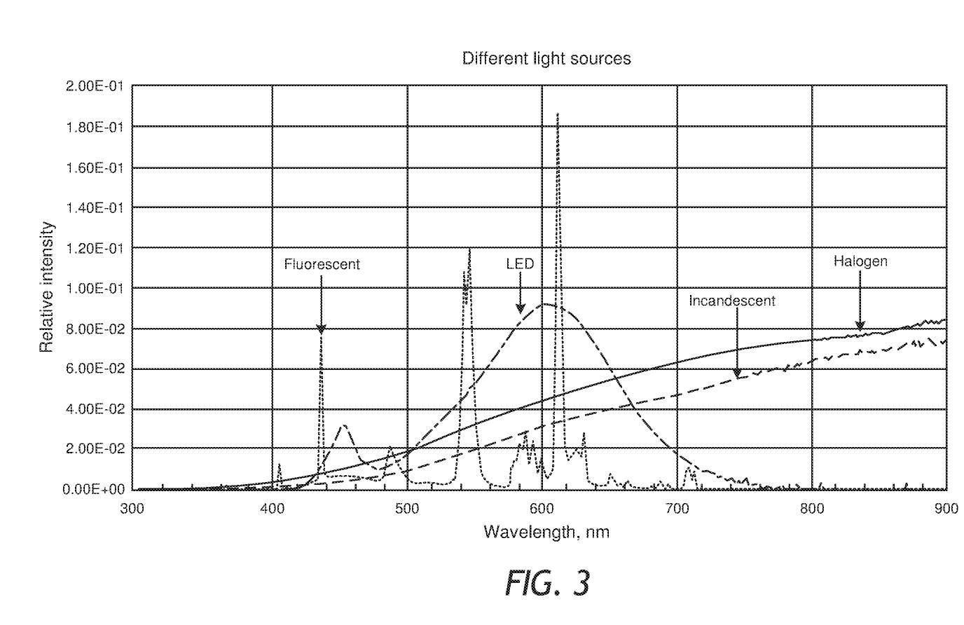

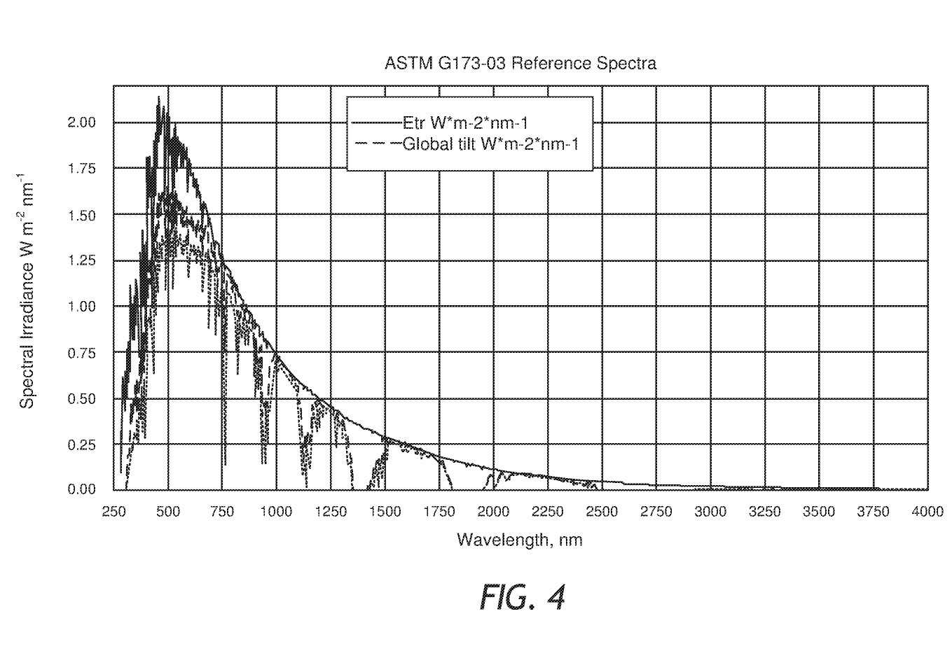

The plasmonic noble metal nanostructures of the present invention can typically have an aspect ratio of greater than 1. The shapes of such nanostructures can be ovals, rectangles, rods, pyramids, random, etc., with rod-shaped nanostructures being preferred (i.e., nanorods, which can also be referred to as nanowires). The general shape of a nanorod is that it has an aspect ratio of greater than 1 and has a generally stick-like or dowel-like shape. Nanorods can be generally straight in the longitudinal direction but can also have some curvature or bend in the longitudinal direction. The aspect ratios of the nanostructures of the present invention can be tuned or designed to be greater than 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9., 3., 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4,9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14,9, 15.0 or more, or any range therein. These various aspect ratios can be used to ensure that the nanostructures respond to desired or targeted light sources. By way of example, and with reference to FIG. 2, the light spectrum and relative intensity of commercial LED lights (Philips A19 60W 2700K and Cree TW T8 2700K) and a fluorescent light source are provided. The primary intensity peaks of the aforementioned commercially available LED lights are around 600 nm to 630 nm. The fluorescent light has an intensity peak of around 620 nm. It has been discovered that an aspect ratio of about 1.7 to about 2.5, preferably about 1.9 to about 2.3, can be used to produce a plasmonic noble metal nanostructure that exhibits a plasmonic peak within this 600 nm to 630 nm range, thereby making such a nanostructure tuned to produce an LSPR in response to such lighting sources. FIG. 3 displays general light intensity versus wavelength relationships for different lights resources. This can be used as guidance in illustrating how to selectively tune a nanostructure of the present invention to a given light source. Further, and with reference to FIG. 4, which concerns the natural light spectrum, a nanostructure having a plasmonic peak of 600 nm to 630 nm can also be capable of producing an LSPR in response to solar radiation (e.g., natural sunlight, an incandescent light source, or a halogen light source or light from a solar simulator) Still further, it has been discovered that an aspect ratio of 2.5 to 3.5, preferably about 3.0, can be used to produce a plasmonic nanostructure having a plasmonic peak within 651 nm to 750 nm, preferably about 675 nm to 725 nm, or more preferably about 700 nm, which can be beneficial for producing an LSPR in response to natural sunlight or light produced from a solar simulator. Similarly, it has been discovered that an aspect ratio of 3.5 to 5.0, preferably about 4.5, can be used to produce a plasmonic nanostructure having a plasmonic peak within 751 nm to 900 nm, preferably about 850 nm, which can also be beneficial for producing an LSPR in response to natural sunlight or light produced from a solar simulator. Even further, is has been discovered that an aspect ratio of: 1) 5.0 to 7.5, preferably 6.0 to 7.0, can be used to produce a plasmonic nanostructure having a plasmonic peak within 901 to 1150 nm, preferably about 1000 nm to 1100 nm in response to electromagnetic radiation; 2). 7.5 to 10.2, preferably 8.0 to 9.2, can be used to produce a plasmonic nanostructure having a plasmonic peak within 1151 to 1400 nm, preferably about 1200 nm to 1300 nm in response to electromagnetic radiation; and 3) 10.2 to 14.5, preferably 11.2 to 12.5, can be used to produce a plasmonic nanostructure having a plasmonic peak within 1401 to 1800 nm, preferably about 1500 nm to 1600 nm in response to electromagnetic radiation. Therefore, use of these combinations of such nanostructures in topical skin formulations can result in a formulation that can be capable of producing LSPRs in various indoor lighting conditions and/or outdoor lighting conditions.

Plasmonic noble metal nanostructures such as nanorods can be purchased from a variety of commercial sources. For example, Sigma-Aldrich® Co. LLC, St. Louis, Mo. (USA) and Nanopartz Inc., Loveland Colo. (USA), each offer gold, platinum, and palladium nanorods in different lengths.

In addition to purchasing plasmonic noble metal nanostructures, there are many techniques known to those having skill in the art for making such nanostructures. By way of example, U.S. Pat. No. 7,691,176 to Niidome et al., which is incorporated by reference, explains that conventional methods of making plasmonic metal nanorods (e.g., gold nanorods) include an electrolytic method, a chemical reduction method, or a photo-reduction method. With the electrolytic method (See, for example, Yu et al., Phys. Chem. B, 1997, 101, p. 6661) a solution containing a cationic surfactant can be electrolyzed by constant current, and gold clusters can be leached from a gold plate at the anode, thereby generating gold nanorods. The surfactant that can be used can be a quaternary ammonium salt having a structure in which four hydrophobic substituents are bonded to a nitrogen atom. Additionally, a compound (e.g., tetradodecylammonium bromide (TDAB)) in which an autonomous molecular assembly is not formed can be added to the solution. When gold nanorods are being produced, the source of the gold can be gold clusters that are leached from a gold plate at the anode. Ultrasonic waves can be radiated during electrolysis, a silver plate can be immersed in the solution, and the growth of the gold nanorods can be accelerated.

By comparison, the chemical reduction method (See, Jana et al., J. Phys. Chem. B, 2001, 105, p. 4065) can include reduction of chlorauric acid with NaBH₄ to generate gold nanoparticles. These nanoparticles, which are considered “seed particles,” can then be grown in solution to produce nanorods. The length of the gold nanorods can be determined according to the quantitative ratio of the “seed particles” to the chlorauric acid added to the growth solution. With the chemical reduction method, it can be possible to generate longer gold nanorods in comparison with the above-described electrolytic method. For this chemical reduction method, two reaction baths for the preparation and reaction to grow the “seed particles” can be used. Chemical reduction is also known as “seeded growth” method can be used for preparing gold nanorods as it produces high yields of rods. Gold nanorods with desired aspect ratios can be obtained by justifying the rod growth condition, including surfactant (CTAB, for instance) and its concentration, silver ion (AgNO₃ for instance) content, temperature and duration, as well as solvent used.

With respect to the photo-reduction method (See, Kim et al., Am. Chem. Soc., 2002, Vol. 124, p. 143160 chloroauric acid can be added to a solution that can be similar to the solution used in the electrolytic method. Ultraviolet irradiation can reduce the chloroauric acid. For irradiation, a low-pressure mercury lamp can be used. In the photo-reduction method, gold nanorods can be generated without producing seed particles. Further, the length of the gold nanorods can be controlled by the irradiation time, with shorter times correlating to shorter rods, and longer times correlating to longer rods.

For each production method, the desired plasmonic noble-metal or precursor material thereof (e.g., noble metal salt) can be used. In some particular embodiments of the present invention, the plasmonic noble-metal nanostructures include noble metals selected from gold, silver, palladium, platinum, or alloys thereof In preferred instances, gold or silver can be used, with gold being more preferable. Further, the produced nanostructures of the present invention can be mixed into and/or dispersed in aqueous or hydrophobic phases of any given topical skin composition and remain stable. This allows for the inclusion of the plasmonic noble metal nanostructures of the present invention in all types of topical skin compositions, including the non-limiting compositions discussed throughout the specification and directly below.

### B. Topical Skin Compositions

The plasmonic noble metal nanostructures of the present invention can be incorporated into topical skin compositions of the present invention. This provides the compositions with the ability to produce LSPR(s) in response to light sources based on the plasmonic peaks of the nanostructures present within the composition. Therefore, topical skin compositions can be designed to produce LSPRs irrespective of whether the end user is present in an environment that largely includes indoor or artificial lighting or in an environment that largely includes solar radiation such as in an outdoor setting. Stated another way, the compositions of the present invention can be tuned to deliver a desired LSPR response for indoor and/or outdoor applications, which can be beneficial in providing the end-user with continuous therapeutically effective LSPR responses in instances where the end user is indoor (e.g., home or office) and moves to the outdoors, and vice versa, or changes a given lighting source (e.g., LED to incandescent lighting, and vice versa).

1. Structure of Compositions

The topical compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, masks, peels, and ointments. Variations and other structures will be apparent to the skilled artisan and are appropriate for use in the present invention. Such structures can be prepared by known techniques using standard mixing, heating, and/or cooling steps. By way of example only, the plasmonic noble metal nanostructures can be incorporated into an existing topical skin composition by simple mixing procedures. This would allow the nanostructures to be dispersed throughout the composition. Generally, selected nanostructures of the present invention can be added to an existing formulation under standard mixing procedures, thereby dispersing the nanostructures throughout the existing formulation. A standard in-tank batch mixer (e.g., high shear disperser and low shear agitators) offered by Admix, Inc., Londonderry, N.H. (USA)), can be used. Alternatively, the plasmonic noble metal nanostructures can be incorporated into any given topical skin composition during the preparing of the composition. By way of example, an oil-in-water emulsion-based topical skin composition includes a continuous aqueous phase and a dispersed hydrophobic phase. The stability of such emulsions is typically derived from the use of surfactants, which can work to maintain the existence of the continuous and discontinuous phases and reduce the occurrence of phase separation. Non-limiting examples of such surfactants that can be used in the context of the present invention include nonionic, cationic, anionic, and zwitterionic emulsifiers. By way of example, McCutcheon's Emulsifiers & Detergents 1986 describes a wide variety of non-limiting surfactants that can be used in the context of this invention. Specific non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, and soaps. During the preparing of such an emulsion, it is typical to produce the aqueous and oil phases in separate containers followed by mixing the two phases in the presence of a surfactant to obtain said emulsion. The ingredients in such phases can be standard cosmetic ingredients known and used in the cosmetics industry, non-limiting examples of which are provided below. The nanostructures of the present invention can be added to the aqueous and/or to the oily phase prior to mixing the phases together. The phases can then be mixed together under typical mixing, heating, and/or cooling conditions to create the emulsion. Similar, mixing, heating, and/or cooling steps can be used to make other formulations such as those noted above (e.g., creams, lotions, solutions (both aqueous and hydro-alcoholic), anhydrous bases (such as lipsticks and powders), gels, masks, peels, and ointments).

2. Cosmetic Ingredients

The topical skin compositions of the present invention can include a variety of cosmetically acceptable ingredients. By way of example, the CTFA International Cosmetic Ingredient Dictionary and Handbook (2004 and 2008 versions) describes a wide variety of non-limiting cosmetic ingredients that can be used in the context of the present invention. Examples of these ingredients include: fragrance agents (artificial and natural; e.g., gluconic acid, phenoxyethanol, and triethanolamine); dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11); flavoring agents/aroma agents (e.g., Stevia rebaudiana (sweetleaf) extract, and menthol); adsorbents; lubricants; solvents; moisturizers (including, e.g., emollients, humectants, film formers, occlusive agents, and agents that affect the natural moisturization mechanisms of the skin); water-repellants; UV absorbers (physical (e.g., titanium dioxide, zinc oxide) and chemical absorbers (e.g., avobenzone, octocrylene, oxybenzone, homosalate, para-aminobenzoic acid (“PABA”), etc.); essential oils; vitamins (e.g., A, B, C, D, E, and K); trace metals (e.g., zinc, calcium and selenium); anti-irritants (e.g., steroids and non-steroidal anti-inflammatories); botanical extracts (e.g., Aloe vera, chamomile, cucumber extract, Ginkgo biloba, ginseng, rosemary, etc.); anti-microbial agents, antioxidants (e.g., BHT and tocopherol); chelating agents (e.g., disodium EDTA and tetrasodium EDTA); preservatives (e.g., methylparaben and propylparaben); pH adjusters (e.g., sodium hydroxide and citric acid); absorbents (e.g., aluminum starch octenylsuccinate, kaolin, corn starch, oat starch, cyclodextrin, talc, and zeolite); skin bleaching and lightening agents (e.g., hydroquinone and niacinamide lactate); humectants (e.g., sorbitol, urea, methyl gluceth-20, saccharide isomerate, and mannitol); exfoliants, waterproofing agents (e.g., magnesium/aluminum hydroxide stearate); skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, dipotassium glycyrrhizate, etc.), silicone containing compounds (e.g., polyorganosiloxanes such as dimethicone, cyclomethicone, polysilicone-11, phenyl trimethicone, trimethylsilylamodimethicone, stearoxytrimethylsilane, etc.), and mixtures thereof.

3. Amounts of Ingredients

The topical skin compositions of the present invention can include any amount of the plasmonic noble metal nanostructures, cosmetic ingredients, and/or other ingredients discussed in this specification. The amounts of such ingredients within the compositions can vary depending on the desired effects of the compositions, its tactile properties, its structure, etc. In non-limiting embodiments, the compositions can include, consist essentially of, or consist of, in their final form, for example, at least about 0.0001%, 0.001%, 0.01%, 0.5%, 1%, 2%, 3%, 4%, 5%, 6%, 7%, 8%, 9%, 10%, 15%, 20%, 25%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition.

4. Products

The compositions of the present invention can be incorporated into a variety of cosmetic products. Non-limiting examples of such products include sunscreen products, sunless skin tanning products, hair products (e.g., shampoos, conditioners, colorants, dyes, bleaches, straighteners, and permanent wave products), fingernail products, moisturizing creams, skin creams and lotions, softeners, day lotions, gels, ointments, foundations, night creams, lipsticks and lip balms, cleansers, toners, masks, deodorants, antiperspirants, exfoliating compositions, shaving-related products (e.g., creams, “bracers” and aftershaves), pre-moistened wipes and washcloths, tanning lotions, bath products such as oils, foot care products such as powders and sprays, skin colorant and make-up products such as foundations, blushes, rouges eye shadows and lines, lip colors and mascaras, baby products (e.g., baby lotions, oils, shampoos, powders and wet wipes), and skin or facial peel products. Additionally, the cosmetic products can be formulated as leave-on or rinse-off products.

### C. Use of Topical Skin Compositions

The topical skin care compositions of the present invention can be used to provide benefits to skin. By way of example, the compositions can be applied to skin (e.g., the face, the neck, the arms, the hands, the chest, the abdomen, the back, the legs, and/or the feet). When the appropriate type of light contacts the composition, the plasmonic nanostructures can absorb light having a certain energy level/wavelength and produce an LSPR response. As explained throughout this specification, the nanostructures can be designed to produce an LSPR(s) in response to artificial light or solar radiation such as natural light or light from a solar simulator. This allows the composition to be designed to work in a targeted lighting environment (e.g., the office environment, the home environment, outdoors, or any combination thereof). Without wishing to be bound by theory, it is believed that the produced LSPR(s) response can be beneficial to skin. By way of example, it is believed that the produced LSPR(s) can reduce the presence of free-radicals and/or oxidants present on the surface of the skin, present within various layers of the skin (e.g., epidermal layer, dermal layer, and/or hypodermal layer), and/or present in various skin cells (e.g., keratinocytes, melanocytes, Merkel cells, and/or Langerhans cells). Again, and without wishing to be bound by theory, it is believed that any given LSPR response can aid in neutralizing the free-radical or oxidative properties of a given free-radical or oxidant by neutralizing unpaired electrons present on the free-radical molecule. By neutralizing these unpaired electrons, the damaging effects of free-radicals on cellular components of the skin such as structural proteins (e.g., collagen, elastin, fibronectin, and laminin), cell membranes, etc., can be reduced. Therefore, it is believed that the compositions of the present invention can help treat, formation of fine lines or wrinkles, sun-damaged skin, skin-inflammatory conditions, skin aging (dark) spots, non-elastic or loosened skin caused by reduced presence of structural proteins, and other skin conditions that may be associated with oxidative damage to skin.

Still further, the compositions of the present invention can be used to treat skin conditions associated with dry skin, itchy skin, flaky skin, inflamed skin, erythemic skin, pain associated with erythemic skin, sensitive skin, pruritus, spider veins, lentigo, age spots, senile purpura, keratosis, melasma, blotches, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, postules, nodules, whiteheads, blackheads, impetigo, erysipelas, erythrism, eczema, sun burns, burned skin, open wounds, skin- inflammatory skin conditions, etc. Even further, the compositions of the present invention through the LSPR activation can be used to (1) reduce tyrosinase activity, TNF-α activity, lipoxygenase activity, and/or matrix metallopeptidase (MMP) (e.g., MMP-1, MMP-3, and/or MMP-9) activity in skin cells and/or (2) activate or increase production of collagen, elastin, fibronectin, and/or laminin in skin. By way of example, reduction in tyrosinase activity can be used to treat hyper-pigmented skin, uneven skin, melasmic skin, dark spots, aged spots, sun spots, blotchy skin, etc. Reduction of TNF-α activity can be used to treat inflamed skin, red skin, erythemic skin, sun burned skin, burned skin, or other skin-related diseases that are also inflammatory diseases. Reduction in lipoxygenase activity and MMP activity can be used to maintain or reduce collagen breakdown in skin cells and can be used to treat (reduce the appearance of) fine lines and wrinkles, sagging skin, loose skin, or non-elastic skin. Activation or upregulation of collagen, elastin, fibronectin, and/or laminin production in skin can also be used to treat (reduce the appearance of) fine lines and wrinkles, sagging skin, loose skin, or non-elastic skin.

All of the compositions and methods disclosed and claimed in this specification can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit, and scope of the invention.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Example 1

### Prophetic Method of Making Plasmonic Noble Metal Nanoparticles

Seeded growth is the major method for producing gold nanorods. The sub 2 nm seed is synthesized by adding HAuCl₄ (0.01 M) into a CTAB (0.1 M) solution. After mixing for 30 minutes, chilled NaBH₄ will be added under vigorous stirring. Then the mixture will be heated gently for about 20 minutes at 60° C. to obtain the seed solution. The growth solution will be include 0.1 M CTAB, 0.01 M HAuCl₄, 0.01 M AgNO₃, and 0.1 M Ascorbic acid. The seed solution will be added to the growth solution to obtain gold nanorods. The Aspect ratio will be tuned by adjusting the conditions of the growth solution; including the CTAB concentration, silver ion concentration, HAuCl₄ concentration, growth temperature and duration, and solvent. Potential suppliers of nanoparticles are Nanopartz Inc., Loveland, Colo. (USA), NanoComposix, Inc. San Diego, Calif. (USA) and Sona Nanotech Ltd (Canada).

### Prophetic Example 2

### Blends of Plasmonic Noble Metal Nanoparticles

The following procedure can be followed to identify appropriate plasmonic noble metal nanoparticle blends or combinations (e.g., combinations of gold nanorods having different plasmonic peaks) that can be used in the topical skin compositions of the present invention. First, a selection of specific gold nanorods having different aspect ratios can be identified. As discussed above, the differing aspect ratios can result in different plasmonic peaks, with larger aspect ratios resulting in plasmonic peaks that have a longer wavelength and smaller aspect ratios resulting in plasmonic peaks having a shorter wavelength. Second, evaluation of the gold nanorods can be performed by comparing the areas under the absorbance peaks of the nanorods vis-à-vis a gold spherical nanoparticle. By way of example, FIG. 1 provides an absorbance comparison of gold (Au) nanospheres (NPS) having a diameter of 30 nm with gold (Au) nanorods (NRS) having varying aspect ratios ranging from 2.8 (10 mm×28 mm), 3.71 (7 nm×26 nm), and 4.5 (8 nm×36 nm). The absorbance was normalized to that of a 30 nm NPS at 400 nm. Comparing to the 30 nm Au NPS, the plasmonic peak areas are 2.07 times for the NRS having an aspect ratio of 2.8, and 2.67 times for the NRS having an aspect ratio of 3.71. FIG. 1 confirms that the plasmonic peaks can be tuned with varying the aspect ratio of a given nanorod. By considering the spectra of indoor light sources and outdoor/natural sunlight, blends of plasmonic nanorods can be developed for cosmetic applications such that an LSPR can be produced by a targeted/selected light source. FIGS. 2 to 4 provide lamp spectra for indoor lighting sources (e.g., fluorescent, LED, incandescent and halogen) and the standard solar spectrum at air mass (AM) 1.5, respectively. It is hypothesized that the plasmonic nanorods having a plasmonic peak at about 600 nm to 630 nm can be used for indoor lighting applications. For outdoor lighting applications (e.g., sunlight or solar simulators), a combination of plasmonic nanorods having plasmonic peaks at about 600 nm, 700 nm, and 850 nm, can be used.

### Prophetic Example 3

### Preparation of a Topical Skin Care Composition and Efficacy of the Composition

The following includes an in vitro bioassay that can be used to measure the anti-oxidant capacity of any one of the plasmonic noble metal nanostructures or combinations of such nanostructures disclosed in the specification. The assay relies on the ability of a sample formulation having the nanostructures of the present invention to inhibit the oxidation of ABTS® (2,2′-azino-di-[3-ethylbenzthiazoline sulphonate]) to ABTS®+by metmyoglobin. The capacity of the nanostructures present in the formulation to prevent ABTS oxidation is compared with that of Trolox, a water-soluble tocopherol analogue, and is quantified as molar Trolox equivalents. Anti-Oxidant capacity kit # 709001 from Cayman Chemical (Ann Arbor, Mich. USA) can be used for this in vitro bioassay. The protocol can be followed according to manufacturer recommendations. A light source can be used that would trigger an LSPR response from the nanostructures (e.g., natural sunlight, a solar simulator, an LED light source, a fluorescent light source, a halogen light source, a laser light source, or an incandescent light source, etc.) depending on the plasmonic peaks of the nanostructures being tested.

1. A topical skin composition comprising:
(a) a plurality of first plasmonic noble metal nanostructures having an aspect ratio of greater than 1 and being configured to absorb electromagnetic radiation from an artificial light source and/or from a solar radiation source to produce a first localized surface plasmon resonance (LSPR); and
(b) a plurality of second plasmonic noble metal nanostructures having an aspect ratio of greater than 1 and being configured to absorb electromagnetic radiation from a solar radiation source to produce a second LSPR.

2. The topical skin composition of claim 1, wherein:
the plurality of first plasmonic noble metals are configured to absorb electromagnetic radiation having a wavelength of 580 nm to 650 nm to produce the first LSPR; and
the plurality of second plasmonic noble metals are configured to absorb electromagnetic radiation having a wavelength of 651 nm to 750 nm to produce the second LSPR.

3. The topical skin composition of claim 2, wherein:
the plurality of first plasmonic noble metals are configured to absorb electromagnetic radiation having a wavelength of 600 nm to 630 nm to produce the first LSPR; and
the plurality of second plasmonic noble metals are configured to absorb electromagnetic radiation having a wavelength of 675 to 725 to produce the second LSPR.

4. The topical skin composition of claim 2, wherein:
the aspect ratio of the plurality of first plasmonic noble metal nanostructures is 1.7 to 2.5; and
the aspect ratio of the plurality of second plasmonic noble metal nanostructures is 2.5 to 3.5.

5. The topical skin composition of claim 1, further comprising a plurality of third plasmonic noble metal nanostructures having an aspect ratio of greater than 1 and being configured to absorb electromagnetic radiation having a wavelength of 751 to 900 nm to produce a third LSPR.

6. The topical skin composition of claim 5, wherein the aspect ratio of the plurality of third plasmonic noble metal nanostructures is 3.5 to 5.0.

7. The topical skin composition of claim 1, further comprising:
a plurality of fourth plasmonic noble metal nanostructures having an aspect ratio of greater than 1 and being configured to absorb electromagnetic radiation having a wavelength of 901 to 1150 nm, preferably about 1000 nm to 1100 nm, to produce a fourth LSPR, wherein the aspect ratio of the plurality of fourth plasmonic noble metal nanostructures is 5.0 to 7.5;
plurality of fifth plasmonic noble metal nanostructures having an aspect ratio of greater than 1 and being configured to absorb electromagnetic radiation having a wavelength of 1151 to 1400 nm, to produce a fifth LSPR, wherein the aspect ratio of the plurality of fifth plasmonic noble metal nanostructures is 7.5 to 10.2;
a plurality of sixth plasmonic noble metal nanostructures having an aspect ratio of greater than 1 and being configured to absorb electromagnetic radiation having a wavelength of 1401 to 1800 nm to produce a sixth LSPR, wherein the aspect ratio of the plurality of sixth plasmonic noble metal nanostructures is 10.2 to 14.5; or
combinations thereof.

8. The topical skin composition of claim 1, wherein the plurality of first plasmonic noble metal nanostructures are configured to absorb electromagnetic radiation from an artificial light source selected from a fluorescent light source, a light emitting diode light source, or a laser source, or a mixture thereof.

9. The topical skin composition of claim 1, wherein the solar radiation source is natural sunlight, a solar simulator, an incandescent light source, or halogen light source.

10. The topical skin composition of claim 1, wherein the plurality of first, second, third, fourth, fifth, and/or sixth plasmonic noble metal nanostructures are gold, silver, or platinum plasmonic nanostructures, or alloys thereof, preferably plasmonic gold nanostructures.

11. The topical skin composition of claim 1, wherein the plurality of first, second, third, fourth, fifth, and sixth plasmonic noble metal nanostructures are nanorods.

12. The topical skin composition of claim 11, wherein the first, second, third, fourth, fifth and/or sixth LSPR(s) are produced along the longitudinal axis of the first, second, third, fourth, fifth and/or sixth plasmonic noble metal nanostructures, respectively.

13. The topical skin composition of claim 1, wherein the composition is an emulsion, preferably an oil-in-water emulsion.

14. The topical skin composition of claim 1, wherein the composition is a cream, a lotion, a solution, a gel, a suspension, or an anhydrous powder or stick.

15. The topical skin composition of claim 1, wherein the composition comprises 0.0001 wt. % to 20 wt. %, of the first, second, third, fourth, fifth and/or sixth plurality of plasmonic noble metal nanostructures, based on the total weight of the composition.

16. A method comprising topically applying the composition of claim 1 to skin.

17. The method of claim 16, wherein the composition is subjected to electromagnetic radiation from:
an artificial light source and/or from a solar radiation source and the plurality of first plasmonic noble metal nanostructures produces a first LSPR; and/or
a solar radiation source and the plurality of second plasmonic noble metal nanostructures produces a second LSPR, the plurality of third plasmonic noble metal nanostructures produces a third LSPR, the plurality of fourth plasmonic noble metal nanostructures produces a fourth LSPR, the plurality of fifth plasmonic noble metal nanostructures produces a fifth LSPR, and/or the plurality of sixth plasmonic noble metal nanostructures produces a sixth LSPR,
wherein the produced first and/or second and/or third LSPR(s) reduce(s) free radicals and/or oxidants.

18. The method of claim 16, wherein:
the aspect ratio of the plurality of first plasmonic noble metal nanostructures is 1.7 to 2.5 and wherein the nanostructures absorb electromagnetic radiation having a wavelength of 580 nm to 650 nm to produce the first LSPR; and/or
the aspect ratio of the plurality of second plasmonic noble metal nanostructures is 2.5 to 3.5 and wherein the nanostructures absorb electromagnetic radiation having a wavelength of 651 nm to 750 nm to produce the second LSPR;
the aspect ratio of the plurality of third plasmonic noble metal nanostructures is 3.5 to 5.0 and wherein the nanostructures absorb electromagnetic radiation having a wavelength of 751 nm to 900 nm to produce the third LSPR;
the aspect ratio of the plurality of fourth plasmonic noble metal nanostructures is 5.0 to 7.5 and wherein the nanostructures absorb electromagnetic radiation having a wavelength of 901 nm to 1150 nm to produce the fourth LSPR;
the aspect ratio of the plurality of fifth plasmonic noble metal nanostructures is 7.5 to 10.2, and wherein the nanostructures absorb electromagnetic radiation having a wavelength of 1151 nm to 1400 nm to produce the fifth LSPR; and/or
the aspect ratio of the plurality of sixth plasmonic noble metal nanostructures is 10.2 to 14.5, and wherein the nanostructures absorb electromagnetic radiation having a wavelength of 1401 nm to 1800 nm to produce the sixth LSPR.

19. The method of claim 16, wherein the artificial lighting source is a fluorescent light source, a light emitting diode light source, or a laser source, or a mixture thereof.

20. The method of claim 16, wherein the solar radiation source is natural sunlight, a solar simulator, an incandescent light source, or a halogen light source.
